(19) United States
(12) Patent Application Publication
(10) Pub. No.: US 2014/0081222 A1
(43) Pub. Date: Mar. 20, 2014

(54) ANTISEPTIC APPICATOR HAVING HYDROPHILIC FOAM FOR USE WITH ANTISEPTIC SOLUTIONS

(71) Applicants: James Patrick McDonald, El Paso, TX (US); Hugo Chavez, El Paso, TX (US); Kenneth M. Dokken, El Paso, TX (US); James Bardwell, El Paso, TX (US)

(72) Inventors: James Patrick McDonald, El Paso, TX (US); Hugo Chavez, El Paso, TX (US); Kenneth M. Dokken, El Paso, TX (US); James Bardwell, El Paso, TX (US)

(73) Assignee: CAREFUSION 2200, INC., San Diego, CA (US)

(21) Appl. No.: 13/620,542

(22) Filed: Sep. 14, 2012

(51) Int. Cl. A61M 35/00

(52) U.S. Cl. CPC A61M 35/003
USPC 604/290; 401/196

(57) ABSTRACT

An applicator having a hydrophilic foam that is suitable for use with antiseptic solutions may be a hydrophilic polyurethane foam that does not reduce the amount of active ingredient in the solution as it passes through the foam so that it can be applied to the skin. The foam may be used to deliver antiseptics solutions such as octenidine salts, chlorhexidine salts, or any cationic molecule to the skin. The foam may be attached to an applicator containing an antiseptic solution, or it may be used in conjunction with an unattached antiseptic solution reservoir. The foam also allows the solution to wick into the foam and fully saturate the foam without dripping or channeling.

### FIELD OF THE INVENTION

The invention relates to an antiseptic applicator having a foam suitable for use with antiseptic solutions and methods for using the antiseptic applicator. The foam may be made of a hydrophilic material that does not reduce the amount of active ingredient in an antiseptic solution as it passes through the foam.

### BACKGROUND OF THE INVENTION

Solutions containing antiseptics, such as octenidine and chlorhexidine, may be applied to the skin to kill bacteria on the skin before performing a medical procedure. Typically, an antiseptic solution is applied to the skin using an antiseptic applicator having a foam pad, which may be attached to the applicator that contains the antiseptic solution. Generally, when an antiseptic solution comes into contact with medical-grade foams, the active component of the solution, such as octenidine and chlorhexidine, often interacts with the foam and may bond to the foam. This interaction reduces the amount of the active component that is available to kill bacteria on the skin.

Thus, there is a need for an antiseptic applicator having a foam that does not reduce the amount of the available active antiseptic component in the solution.

### SUMMARY OF THE INVENTION

The invention relates to an antiseptic applicator having a hydrophilic foam suitable for use with antiseptic solutions. The foam comprises a hydrophilic polyurethane foam that does not substantially reduce the amount of active ingredient in the solution as it passes through the foam so that it can be applied to the skin. The foam may be used to deliver antiseptics solutions including, but not limited to octenidine salts, chlorhexidine salts, alcohol, aldehyde, anilide, diamidine, halogen-releasing agent, silver compound, peroxygen, phenols, and/or biguanides such as alexidine, phenoxyethanol, bis-phenols, quaternary ammonium compounds, PVP-iodine or any cationic molecule to the skin. The foam may also be used in conjunction with a separate container comprising antiseptic solution instead of being attached to an applicator. The foam also allows the solution to wick into the foam and fully saturate the foam without dripping or channeling.

### DETAILED DESCRIPTION OF THE INVENTION

The invention relates to an antiseptic applicator having a hydrophilic foam suitable for use with antiseptic solutions. The foam comprises a hydrophilic polyurethane foam that does not substantially reduce the amount of active ingredient in the solution as it passes through the foam so that it can be applied to the skin. The foam may be used to deliver antiseptics solutions such as octenidine salts, chlorhexidine salts, alcohol, aldehyde, anilide, diamidine, halogen-releasing agent, silver compound, peroxygen, phenols, and/or biguanides such as alexidine, phenoxyethanol, bis-phenols, quaternary ammonium compounds, PVP-iodine or any cationic molecule to the skin.

Preferably, the solution is an aqueous solution, for example containing 0.001%-10% of antiseptic. When the antiseptic solution comes in contact with the foam, the properties of the foam allow the solution to quickly wick into the foam and fully saturate the foam to create a reservoir of solution.

According to an embodiment of the invention, the foam may comprise a hydrophilic, non-swelling, biocompatible foam. A preferred hydrophilic foam that may be used in this invention is the SAQ-100 foam sold by the INOAC Foam Company. The hydrophilic foam may have one or more of the following qualities: a density of 85-115 kg/m³, a hardness of 145-170 N, a tensile strength of 210-250 kPa, an elongation of 200-300%, 65-90 pores per inch (PPI), a wicking rate of 0.5-3.5 sec, and an expansion of less than 5%. According to the invention, this foam may be used with an aqueous antiseptic solution preferably containing an active ingredient such as octenidine dihydrochloride or chlorhexidine gluconate.

The foam is configured to allow 90% or more of the antiseptic to remain in the solution after the solution passes through the hydrophilic foam member, preferably 95% or more, and most preferably 98% or more.

A foam according to the invention may be cut into various shapes and adapted to fit a delivery device. For example, a foam may be used as an applicator for a surgical preparation applicator used to prepare a surgical site or placed over a wound to promote healing. The antiseptic solution may be contained in a reservoir, such as a frangible ampoule, inside the applicator.

An applicator according to the invention may comprise a head portion and a container portion. The head portion includes a proximal end, a distal end, and an interior portion defining a fluid chamber. The container portion may include a reservoir that contains the antiseptic solution. The foam member may be attached to the distal end. The foam and antiseptic solution of the invention may be implemented in any suitable applicator apparatus. Some examples of suitable applicators include: U.S. Pat. Nos. 3,757,782; 4,415,288; 4,498,796; 5,288,159; 5,308,180; 5,435,660; 5,445,462; 5,658,084; 5,772,346; 5,791,801; 5,927,884; 6,371,675; 5,769,552; 6,488,665; 6,916,133; 7,201,525; and 7,866,907, each hereby incorporated by reference herein.

Alternatively, the foam of the invention may be used in conjunction with unattached separate container comprising antiseptic solution.

When applying an antiseptic solution to the skin, it may be desirable to include a dye with the antiseptic solution so that the areas of the skin contacted with the antiseptic solution may be visible to a user. This dye may be incorporated into the antiseptic solution, or it may be incorporated into a pledget, which is a piece of material typically made of a foam or a laminate. In an embodiment of the invention, the foam may be used with an applicator that includes a pledget containing a dye. As the antiseptic solution flows out of its reservoir in the applicator, the antiseptic solution passes through the pledget, and the antiseptic solution and dye pass on to the foam. Examples of such an arrangement can be found in U.S. Pat. No. 7,866,907 and U.S. application Ser. No. 12/961,255, filed on Dec. 6, 2010, each hereby incorporated by reference.

In accordance with the present specification, the term “substantially” means preferably about 10% or less, more preferably about 5% or less.

Experiments were performed to compare the performances of various types of foam when contacted with an aqueous solution containing 0.4% octenidine hydrochloride and 5% w/v polyethylene glycol (PEG). The experiments measured physical flow characteristics and the percent loss of octenidine after the antiseptic solution passed through the foam. These experiments are summarized in Examples 1 and 2 below.

### Example 1

### Foams Tested without Applicators and Pledgets

The foams tested were INOAC SAQ-type (SAQ-75, INOAC SAQ-100, INOAC SAQ-3x Press, INOAC SAQ-2.5x Press), INOAC HC type (INOAC HC 1½ Press, and INOAC HC 2⅓ Press), and Foamex International Inc (FXI)-type (FXI-10, FXI-11, FXI-12, FXI-13, FXI-14, FXI-15, FXI-16, and FXI-17). FXI-type foam (specifically part #FLM141) comprises a natural color non-pigment polyester urethane. The FXI foam has a pore size of 55-85 ppi, a density of 1.62-2.01 lb/ft³, a tensile strength of 24-40 psi minimum, an elongation 200%-250% minimum, and a laminated bond strength of 16 oz minimum. The INOAC SAQ-type foam comprises a plastic made of hydroxyl compounds (polyol) and isocyanate. INOAC SAQ has a density 75 kg/m³, a hardness 137N, a tensile strength of 77 kPa, a pore count of 74-78 ppi, an elongation of 300%, a wicking rate of 1-2 seconds, an absorption of 8 times, and an expansion 3%. INOAC HC comprises a plastic made of hydroxyl compounds (polyol) and isocyanate. INOAC clear has a density 32.7 kg/m³, a hardness 0.65 psi, a tensile strength of 15 lb/in, a pore count of 62-66 ppi, an elongation of 250%, a wicking rate of 2-3 seconds, an absorption of 18 times, and an expansion 3%. Three samples of each type of foam were tested.

Physical parameters including wet time, drip time, and drain time after activation, or when the antiseptic solution was released from its reservoir, were measured for each of the foams tested. The wet time is the time for the fluid to reach the foam, starting from the time of activation. A foam that achieves a wet time of 10 seconds or less is preferable, and a wet time of 5 seconds or less is more preferable. The drip time is the amount of time after activation at which the foam tip begins to drip. A foam that exhibits no dripping within 15 seconds of activation is preferable. In the experiments, if a foam did not exhibit any dripping within 15 seconds of activation, the drip time was recorded as “no drip”. This time is also referred herein as the dripping termination time. The drain time is the time that takes the applicator to discharge the entire solution into the foam. A foam that exhibits drain time of less than 30 seconds is preferable.

Another test called “extraction” or “milking” was performed by forcing the antiseptic solution through the foam to determine how much of the solution may be extracted from the foam. This test was performed by weighing the applicator containing the antiseptic solution and foam, recording the initial weight of the applicator, placing the applicator inside a glass beaker, activating the applicator by opening the reservoir, waiting 30 seconds for the antiseptic solution to drain into the foam tip, extracting the antiseptic solution by pressing on the foam tip against inner wall of the glass beaker for 30 seconds to force solution out of foam tip, removing the applicator from the glass beaker, weighing the applicator, and recording the final weight of the applicator.

The extraction amount was calculated by subtracting the final weight of the applicator from the initial weight of the applicator. Preferably, the foam achieves an extraction amount evidencing that about 90% or more of the antiseptic solution, more preferably about 95% or more, most preferably about 98% or more, passes through the foam.

The concentration of antiseptic in the solutions before and after passing through the foams was measured by high pressure liquid chromatography (HLPC) performed on an Agilent 1200 HPLC-6 instrument with a Zorbax Eclipse XDB-C18 3.0×150 mm, 5 micron column.

The percent loss of octenidine for each foam, which is based on the ratio of concentration of the octenidine in the solution extracted from the foam and the concentration of the octenidine in the antiseptic solution before passing through the foam, was calculated according to the Formula 1:

$$Loss of Octenidine ( \% ) = 1 - \frac{ \begin{matrix}octenidine concentration in solution \\ extracted from foam\end{matrix}}{ \begin{matrix}octenidine concentration in solution \\ before passing through foam\end{matrix}} \times 100 \qquad Formula 1$$

As each foam sample was tested three times, an average percent loss of octenidine for each type of foam was calculated.

The experimental results are summarized in Table 1 below.

TABLE 1

| | | Wet | Drip | Drain | | Octenidine | Loss of |
|---|---|---|---|---|---|---|---|
| | Thickness | Time | Time | Time | Extraction | Concentration | Octenidine |
| Foam | (in) | (sec) | (sec) | (sec) | (g) | (% w/v) | (%) |
| SAQ- | 0.385 | 3.25 | no drip | 6.04 | 16.33 | 0.37339 | 8.12 |
| 75 | | | | | | | |
| SAQ- | 0.385 | 2.18 | no drip | 5.78 | 19.602 | 0.36436 | |
| 75 | | | | | | | |
| SAQ- | 0.385 | 2.39 | no drip | 5.58 | 19.619 | 0.36502 | |
| 75 | | | | | | | |
| SAQ- | 0.345 | 3.11 | 4.97 | 13.25 | 17.893 | 0.39031 | 2.69 |
| 100 | | | | | | | |
| SAQ- | 0.345 | 1.62 | 1.62 | 5.02 | 17.597 | 0.39008 | |
| 100 | | | | | | | |
| SAQ- | 0.345 | 2.44 | 3.14 | 7.70 | 20.811 | 0.38763 | |
| 100 | | | | | | | |
| SAQ- | 0.375 | 15.02 | no drip | 120 | 15.675 | 0.30796 | 24.15 |
| 3x | | | | | | | |
| Press | | | | | | | |
| SAQ- | 0.375 | 14.84 | no drip | 80 | 14.629 | 0.31144 | |
| 3x | | | | | | | |
| Press | | | | | | | |
| SAQ- | 0.375 | 15.07 | no drip | 145 | 16.306 | 0.29098 | |
| 3x | | | | | | | |
| Press | | | | | | | |
| SAQ- | 0.355 | 9.53 | no drip | 60 | 11.194 | 0.29500 | 15.67 |
| 2.5x | | | | | | | |
| Press | | | | | | | |
| SAQ- | 0.355 | 4.20 | no drip | 30.71 | 17.376 | 0.34632 | |
| 2.5x | | | | | | | |
| Press | | | | | | | |
| SAQ- | 0.355 | 7.17 | no drip | 50.92 | 16.251 | 0.37084 | |
| 2.5x | | | | | | | |
| Press | | | | | | | |
| HC-2 | 0.316 | 1.58 | 2.98 | 9.29 | 18.78 | 0.36734 | 8.02 |
| ½ | | | | | | | |
| Press | | | | | | | |
| HC-2 | 0.316 | 2.28 | 5.96 | 16.37 | 18.60 | 0.36837 | |
| ½ | | | | | | | |
| Press | | | | | | | |
| HC-2 | 0.316 | 1.80 | 2.81 | 9.12 | 19.5 | 0.36828 | |
| ½ | | | | | | | |
| Press | | | | | | | |
| HC-2 | 0.316 | 3.61 | no drip | 71 | 11.65 | 0.31872 | 12.05 |
| ⅓ | | | | | | | |
| Press | | | | | | | |
| HC-2 | 0.316 | 5.31 | no drip | 120 | 15.54 | 0.38946 | |
| ⅓ | | | | | | | |
| Press | | | | | | | |
| HC-2 | 0.316 | 3.05 | 7.95 | 19.5 | 18.40 | 0.34740 | |
| ⅓ | | | | | | | |
| Press | | | | | | | |
| FXI-10 | .32 | 4.34 | No Drip | 6.48 | 20.819 | 0.30000 | 24.00 |
| FXI-10 | .32 | 1.46 | No Drip | 6.14 | 21.674 | 0.30699 | |
| FXI-10 | .32 | 1.17 | No Drip | 6.15 | 20.949 | 0.30526 | |
| FXI-11 | .32 | 2.04 | No Drip | 6.21 | 20.582 | 0.29035 | 28.58 |
| FXI-11 | .32 | 1.52 | No Drip | 5.52 | 20.891 | 0.28716 | |
| FXI-11 | .32 | 1.82 | No Drip | 5.62 | 20.371 | 0.27970 | |
| FXI-12 | .40 | 1.89 | No Drip | 5.61 | 21.729 | 0.29247 | 27.12 |
| FXI-12 | .40 | 2.10 | No Drip | 4.94 | 21.737 | 0.29969 | |
| FXI-12 | .40 | 2.72 | No Drip | 5.97 | 21.682 | 0.28253 | |
| FXI-13 | .40 | 2.44 | No Drip | 5.19 | 20.742 | 0.27673 | 31.15 |
| FXI-13 | .40 | 6.50 | No Drip | 5.77 | 20.030 | 0.27651 | |
| FXI-13 | .40 | 2.41 | No Drip | 5.82 | 21.357 | 0.27315 | |
| FXI-14 | .32 | 1.38 | 7.19 | 5.68 | 20.924 | 0.29117 | 27.88 |
| FXI-14 | .32 | 1.48 | No Drip | 6.44 | 22.007 | 0.29082 | |
| FXI-14 | .32 | 2.00 | No Drip | 6.70 | 22.151 | 0.2836 | |
| FXI-14 | .32 | 2.14 | 5.49 | 7.50 | 20.125 | N/A | |
| FXI-15 | .32 | 1.59 | No Drip | 3.86 | 19.014 | 0.27473 | 31.93 |
| FXI-15 | .32 | 1.50 | No Drip | 4.14 | 20.500 | 0.26964 | |
| FXI-15 | .32 | 1.46 | No Drip | 5.75 | 20.408 | 0.27261 | |
| FXI-16 | .40 | 3.20 | No Drip | 6.07 | 20.929 | 0.29662 | 27.14 |
| FXI-16 | .40 | 3.00 | No Drip | 6.78 | 21.258 | 0.28470 | |
| FXI-16 | .40 | 2.71 | No Drip | 6.48 | 21.159 | 0.29321 | |
| FXI-17 | .40 | 2.43 | No Drip | 5.88 | 20.360 | 0.26231 | 34.92 |
| FXI-17 | .40 | 1.89 | No Drip | 4.95 | 19.309 | 0.25073 | |
| FXI-17 | .40 | 3.72 | No Drip | 6.21 | 20.660 | 0.26814 | |

### Example 2

### Foams Tested with Applicators and Pledgets

The foams tested were the INOAC SAQ-100 foam, the FXI 3X Z80D, FXI 3X EC65, and the FXI 3X EFLTZ851HA foams made by Foamex International Inc (FXI). Five samples of each type of foam were tested.

Applicators having reservoirs of 10.5 mL and 26 mL sizes were tested. The 10.5 mL applicators had circular-shaped foams having a diameter of 1.5 inches and a thickness of 0.360 inches. The 26 mL applicators had square-shaped foams having a side length of 2.25 inches and a thickness of 0.360 inches.

In the experiments, the applicators included different types of pledgets that were embedded with a dye. In some experiments, 100% non-woven needle-punched polyester pledgets having a fiber weight per unit length of 6 denier were used.

In other experiments, the pledgets were formed of a laminated material formed of Novonette SP-149305, a nonwoven polyester, and SIF #3-1000Z felt, a non-pigmented reticulated polyester urethane. Applicators having these pledgets are denoted as “N” in the tables below.

These tests were performed by opening the reservoir in the applicator by breaking the ampoule, and starting a stopwatch to measure the wet time, drip time, and drain time, as discussed in Example 1 above.

The percent loss of octenidine for each foam was also calculated according to the Formula 1 above.

The experimental results are summarized in Table 2 below.

TABLE 2

| | | | | | | | |
|---|---|---|---|---|---|---|---|
| Applicator | Foam | Wet | Drip | Drip | | | |
| volume | thickness | Time | Time | Time | Extraction | | Loss of |
| (ml) | (mm) | (sec) | (sec) | (sec) | (g) | pH | Octenidine (%) |

Foam: INOAC SAQ-100

| | | | | | | | |
|---|---|---|---|---|---|---|---|
| 10.5 ml | 9.1 | 6.1 | 12.8 | 30.5 | 6.3 | 6.18 | 3.47 |
| 10.5 ml | 9.1 | 1.8 | 2.6 | 5.93 | 7.1 | | |
| 10.5 ml | 9.1 | 3.47 | 4.8 | 8.9 | 6.9 | | |
| 10.5 ml | 9.1 | 5.50 | 6.9 | 11.8 | 6.5 | | |
| 10.5 ml | 9.1 | 2.94 | 4.2 | 9.0 | 7.3 | | |
| 10.5 ml | 10.2 | 3.04 | 4.2 | 6.8 | 7.1 | 6.21 | 4.78 |
| 10.5 ml | 10.2 | 10.42 | 17.8 | 41.7 | 5.9 | | |
| 10.5 ml | 10.2 | 3.17 | 4.4 | 7.9 | 7.3 | | |
| 10.5 ml | 10.2 | 7.19 | 14.7 | 38.8 | 6.9 | | |
| 10.5 ml | 10.2 | 3.62 | 4.48 | 7.8 | 7.5 | | |
| 10.5 ml | 11.4 | 7.08 | 13.1 | 17.8 | 6.9 | 6.22 | 4.88 |
| 10.5 ml | 11.4 | 4.07 | 6.49 | 12.6 | 7.3 | | |
| 10.5 ml | 11.4 | 6.17 | 12.7 | 21.2 | 7.1 | | |
| 10.5 ml | 11.4 | 8.7 | 13.3 | 28.3 | 6.0 | | |
| 10.5 ml | 11.4 | 5.85 | 13.4 | 25.1 | 6.7 | | |
| 10.5 ml N | 8.9 | 1.3 | 4.5 | 7.5 | 6.8 | 6.34 | 5.74 |
| 10.5 ml N | 8.9 | 2.5 | 9.3 | 18.8 | 7.5 | | |
| 10.5 ml N | 8.9 | 1.8 | 6.5 | 12.1 | 7.4 | | |
| 10.5 ml N | 8.9 | 1.9 | 5.2 | 10.7 | 7.2 | | |
| 10.5 ml N | 8.9 | 1.8 | 3.3 | 6.4 | 7.3 | | |
| 10.5 ml N | 9.9 | 3.7 | 4.5 | 6.1 | 7.1 | 6.43 | 6.66 |
| 10.5 ml N | 9.9 | 1.9 | 3.8 | 5.6 | 7.5 | | |
| 10.5 ml N | 9.9 | 1.2 | 3.1 | 4.8 | 7.8 | | |
| 10.5 ml N | 9.9 | 7.0 | 2.7 | 5.1 | 7.5 | | |
| 10.5 ml N | 9.9 | 1.6 | 5.3 | 8.7 | 7.0 | | |
| 10.5 ml N | 11.2 | 2.1 | 7.5 | 16.5 | 7.2 | 6.57 | 7.33 |
| 10.5 ml N | 11.2 | 2.0 | 4.3 | 11.9 | 6.9 | | |
| 10.5 ml N | 11.2 | 2.6 | no | 19.5 | 6.5 | | |
| | | | drip | | | | |
| 10.5 ml N | 11.2 | 2.5 | 6.7 | 21.9 | 7.0 | | |
| 10.5 ml N | 11.2 | 1.3 | no | 7.4 | 6.7 | | |
| | | | drip | | | | |
| 26 ml | 9.1 | .86 | 1.8 | 3.9 | 18.7 | 6.29 | 4.07 |
| 26 ml | 9.1 | 2.9 | 3.7 | 11.1 | 16.9 | | |
| 26 ml | 9.1 | 1.9 | 3.5 | 26.1 | 17 | | |
| 26 ml | 9.1 | 2.6 | 3.9 | 19.5 | 14 | | |
| 26 ml | 9.1 | 1.5 | 2.7 | 15.3 | 17.7 | | |
| 26 ml | 10.2 | 5.8 | 10.1 | 46.6 | 17 | 6.34 | 3.20 |
| 26 ml | 10.2 | 3.0 | 3.84 | 8.2 | 17.4 | | |
| 26 ml | 10.2 | 1.8 | 2.6 | 8.7 | 18.2 | | |
| 26 ml | 10.2 | 1.7 | 2.6 | 5.5 | 17.4 | | |
| 26 ml | 10.2 | 1.9 | 3.1 | 7.7 | 17.6 | | |
| 26 ml | 11.4 | 4.8 | 7.2 | 19.9 | 16.1 | 6.46 | 5.25 |
| 26 ml | 11.4 | 1.6 | 3.1 | 6.7 | 16.4 | | |
| 26 ml | 11.4 | 2.9 | 4.2 | 11.4 | 16.2 | | |
| 26 ml | 11.4 | 4.1 | 5.2 | 9.2 | 17.6 | | |
| 26 ml | 11.4 | 2.5 | 3.5 | 19.0 | 17.7 | | |
| 26 ml N | 8.9 | 1.9 | 3.4 | 19.0 | 18.2 | 6.32 | 6.95 |
| 26 ml N | 8.9 | 1.3 | 2.0 | 7.7 | 19.2 | | |
| 26 ml N | 8.9 | 1.9 | 3.1 | 16.2 | 19.2 | | |
| 26 ml N | 8.9 | 1.3 | 2.5 | 10.7 | 19.6 | | |
| 26 ml N | 8.9 | 1.2 | 2.5 | 15.3 | 18.7 | | |
| 26 ml N | 9.9 | 1.87 | 2.85 | 6.2 | 18.5 | 6.37 | 6.00 |
| 26 ml N | 9.9 | 2.20 | 3.6 | 10.8 | 18.1 | | |
| 26 ml N | 9.9 | 1.26 | 2.2 | 7.9 | 18.8 | | |
| 26 ml N | 9.9 | 1.26 | 2.3 | 7.9 | 19.5 | | |
| 26 ml N | 9.9 | 1.0 | 4.9 | 6.5 | 18.5 | | |
| 26 ml N | 11.2 | 1.5 | 4.9 | 8.6 | 17.4 | 6.65 | 7.20 |
| 26 ml N | 11.2 | 1.6 | 2.6 | 13.2 | 17.7 | | |
| 26 ml N | 11.2 | 1.2 | 8.2 | 28.6 | 16.5 | | |
| 26 ml N | 11.2 | 2.8 | 11.9 | 36.4 | 17.9 | | |
| 26 ml N | 11.2 | 3.1 | 7.7 | 16.9 | 17.9 | | |

Foam: FXI-3X (26 ml)

| | | | | | | | |
|---|---|---|---|---|---|---|---|
| F3-Z80D N | 10.2 | 2.2 | 2.9 | 8.4 | 13.1 | 5.26 | 1.92 |
| F3-Z80D N | 10.2 | 2.9 | 3.4 | 7.7 | 18.8 | | |
| F3-Z80D N | 10.2 | 1.9 | 2.5 | 11.2 | 18.8 | | |
| F3-Z80D N | 10.2 | 3.2 | 3.9 | 11.9 | 18.9 | | |
| F3-Z80D N | 10.2 | 2.7 | 3.5 | 8.7 | 19.3 | | |
| F3-EC65 N | 10.2 | 2.4 | 3.1 | 6.6 | 19.2 | 4.79 | 6.63 |
| F3-EC65 N | 10.2 | 1.8 | 2.4 | 7.2 | 19.3 | | |
| F3-EC65 N | 10.2 | 3.1 | 3.7 | 6.1 | 19 | | |
| F3-EC65 N | 10.2 | 3.2 | 3.8 | 7.1 | 19.5 | | |
| F3-EC65 N | 10.2 | 2.3 | 3.4 | 6.4 | 19.6 | | |
| F3- | 10.2 | 6.4 | 7.2 | 22.7 | 20.4 | 5.38 | 0.95 |
| EFLTZ85IHA N | | | | | | | |
| F3- | 10.2 | 10.8 | 12.2 | 20 | 20.9 | | |
| EFLTZ85IHA N | | | | | | | |
| F3- | 10.2 | 10.3 | 10.9 | 26.9 | 20.6 | | |
| EFLTZ85IHA N | | | | | | | |
| F3- | 10.2 | 12.4 | 14.8 | 24.8 | 21.2 | | |
| EFLTZ85IHA N | | | | | | | |
| F3- | 10.2 | 8.5 | 8.9 | 18.3 | 20.1 | | |
| EFLTZ85IHA N | | | | | | | |

What is claimed is:

1. An applicator assembly comprising:
a head portion having a proximal end, a distal end, and an interior portion defining a fluid chamber;
a container coupled to the head portion and comprising a solution containing an antiseptic; and
a hydrophilic foam member attached to the distal end of the head portion,
wherein the hydrophilic foam member is configured to allow 90% or more of the antiseptic to remain in the solution after the solution passes through the hydrophilic foam member.

2. The applicator of claim 1, wherein the hydrophilic foam member is configured to allow 95% or more of the antiseptic to remain in the solution after the solution passes through the hydrophilic foam member.

3. The applicator of claim 1, wherein the hydrophilic foam member is configured to allow 98% or more of the antiseptic to remain in the solution after the solution passes through the hydrophilic foam member.

4. The applicator of claim 1, wherein the antiseptic is selected from the group consisting of: octenidine salts, chlorhexidine salts, alcohol, aldehyde, anilide, diamidine, halogen-releasing agent, silver compound, peroxygen, phenols, and biguanides.

5. The applicator of claim 4, wherein the solution comprises from 0.001%-10% by volume antiseptic.

6. The applicator of claim 4, wherein the antiseptic comprises octenidine hydrochloride or chlorhexidine gluconate.

7. The applicator of claim 1, wherein the solution comprises an aqueous solution.

8. The applicator of claim 1, wherein the hydrophilic foam member comprises at least one of: a density of 85-115 kg/m³, a hardness of 145-170 N, a tensile strength of 210-250 kPa, an elongation of 200-300%, 65-90 pores per inch, a wicking rate of 0.5-3.5 sec, and an expansion of less than 5%.

9. The applicator of claim 1, wherein the hydrophilic foam member comprises a wet time of 10 seconds or less, a dripping termination time of 15 seconds or less, and a drain time of less than 30 seconds.

10. A solution containing system comprising:
a container comprising a solution containing an antiseptic; and
a hydrophilic foam member,
wherein the hydrophilic foam member is configured to allow 90% or more of the antiseptic to remain in the solution after the solution passes through the hydrophilic foam member.

11. The system of claim 10, wherein the hydrophilic foam member is configured to allow 95% or more of the antiseptic to remain in the solution after the solution passes through the hydrophilic foam member.

12. The system of claim 10, wherein the hydrophilic foam member is configured to allow 98% or more of the antiseptic to remain in the solution after the solution passes through the hydrophilic foam member.

13. The system of claim 10, wherein the antiseptic is selected from the group consisting of: octenidine salts, chlorhexidine salts, alcohol, aldehyde, anilide, diamidine, halogen-releasing agent, silver compound, peroxygen, phenols, and biguanides.

14. The system of claim 13, wherein the solution comprises from 0.001%-10% by volume antiseptic.

15. The system of claim 13, wherein the antiseptic comprises octenidine hydrochloride or chlorhexidine gluconate.

16. The system of claim 10, wherein the solution comprises an aqueous solution.

17. The system of claim 10, wherein the hydrophilic foam member comprises at least one of: a density of 85-115 kg/m³, a hardness of 145-170 N, a tensile strength of 210-250 kPa, an elongation of 200-300%, 65-90 pores per inch, a wicking rate of 0.5-3.5 sec, and an expansion of less than 5%.

18. The system of claim 10, wherein the hydrophilic foam member comprises a wet time of 10 seconds or less, a dripping termination time of 15 seconds or less, and a drain time of less than 30 seconds.

19. A method of treating a surface comprising:
passing a solution containing an antiseptic through a hydrophilic foam member, wherein after passing the solution through the hydrophilic foam member, 90% or more of the antiseptic remains in the solution; and
contacting the solution with a surface.

20. The method of claim 19, wherein after passing the solution through the hydrophilic foam member, 95% or more of the antiseptic remains in the solution.

21. The method of claim 19, wherein after passing the solution through the hydrophilic foam member, 98% or more of the antiseptic remains in the solution.

22. The method of claim 19, wherein the antiseptic is selected from the group consisting of: octenidine salts, chlorhexidine salts, alcohol, aldehyde, anilide, diamidine, halogen-releasing agent, silver compound, peroxygen, phenols, and biguanides.

23. The method of claim 22, wherein the solution comprises from 0.001%-10% by volume antiseptic.

24. The method of claim 22, wherein the antiseptic comprises octenidine hydrochloride or chlorhexidine gluconate.

25. The method of claim 19, wherein the solution comprises an aqueous solution.

26. The method of claim 19, wherein the hydrophilic foam member comprises at least one of: a density of 85-115 kg/m³, a hardness of 145-170 N, a tensile strength of 210-250 kPa, an elongation of 200-300%, 65-90 pores per inch, a wicking rate of 0.5-3.5 sec, and an expansion of less than 5%.

27. The method of claim 19, wherein the hydrophilic foam member comprises a wet time of 10 seconds or less, a dripping termination time of 15 seconds or less, and a drain time of less than 30 seconds.

28. The method of claim 19, wherein the surface comprises skin.
